(19) United States
(12) Patent Application Publication
(10) Pub. No.: US 2023/0167124 A1
(43) Pub. Date: Jun. 1, 2023

(54) Methods for the Production of Nickel (II) Etioporphyrin-I

(71) Applicant: Adgero Biopharmaceuticals Holdings, Inc., San Diego, CA (US)

(72) Inventor: Shwn-Ji S.H. Lee, Princeton, NJ (US)

(73) Assignee: Adgero Biopharmaceuticals Holdings, Inc., San Diego, CA (US)

(21) Appl. No.: 17/614,132

(22) PCT Filed: Oct. 4, 2021
(86) PCT No.: PCT/US2021/053362

§ 371 Date: Nov. 24, 2021

Related U.S. Application Data
(60) Provisional application No. 63/086,759, filed on Oct. 2, 2020.

(51) Int. Cl. C07D 487/22

(52) U.S. Cl. CPC C07D 487/22

(57) ABSTRACT

Improved methods for the production of nickel (II) etioporphyrin-1. The improved method for the production of nickel (ll) etioporphyrin-l comprises brominating kryptopyrrole to provide monobromo-dipyrromethene, wherein the bromination is carried out in the presence of ethyl acetate; (b) cyclizing monobromo-dipyrromethene to provide etioporphyrin-1; and metallating etioporphyrin-1 with a nickel (II) salt to provide nickel (II) etioporphyrin-1.

### CROSS-REFERENCE TO RELATED APPLICATION

This application claims the benefit of U.S. Application No. 63/086,759, filed Oct. 2, 2020, expressly incorporated herein by reference in its entirety.

### FIELD OF THE INVENTION

The present invention relates to methods for the production of dipyrromethene and porphyrinic compounds useful as starting materials for metallated etioporphyrin-1 compounds useful as photosensitizers or as building blocks of photosensitizers in photodynamic therapy (PDT).

### BACKGROUND

Pyrroles, dipyrromethanes, dipyrromethenes, and porphyrins are used extensively as building blocks to, or as photosensitizers in photodynamic therapy (PDT). Photodynamic therapy is a procedure that uses photoselective (light-activated) drugs to target and destroy diseased cells. Photoselective drugs transform light energy into chemical energy in a manner similar to the action of chlorophyll in green plants. The photoselective drugs are inactive until switched on by light of a specific wavelength thereby enabling physicians to target specific groups of cells and control the timing and selectivity of treatment. The result of this process is that diseased cells are destroyed with minimal damage to surrounding normal tissues.

Photodynamic therapy begins with the administration, to a patient, of a preferred amount of a photoselective compound that is selectively taken up and/or retained by the biologic target (e.g., tissue or cells). After the photoselective compound is taken up by the target, a light of the appropriate wavelength to be absorbed by the photoselective compound is delivered to the targeted area. This activating light excites the photoselective compound to a higher energy state. The extra energy of the excited photoselective compound can then be used to generate a biological response in the target area by interaction with oxygen. As a result of the irradiation, the photoselective compound exhibits cytotoxic activity (i.e., it destroys cells). Additionally, by localizing in the irradiated area, it is possible to contain the cytotoxicity to a specific target area. For a more detailed description of photodynamic therapy, see U.S. Pat. Nos. 5,225,433, 5,198,460, 5,171,749, 4,649,151, 5,399,583, 5,459,159, and 5,489,590, the disclosures of which are incorporated herein by reference.

The synthesis of porphyrins from mono-pyrrolic precursors has been studied for decades (for example see “The Porphyrins”, volume I, II, Ed: D. Dolphin, Academic press, 1978) and is the preferred route to symmetrical porphyrins bearing identical β-pyrrolic substituents (for example octaethylporphyrin (1) (Scheme 1)). In this instance acid catalysed tetramerization of monopyrroles results in the formation of an unstable porphyrinogen which is oxidize by air to give the desired porphyrin.

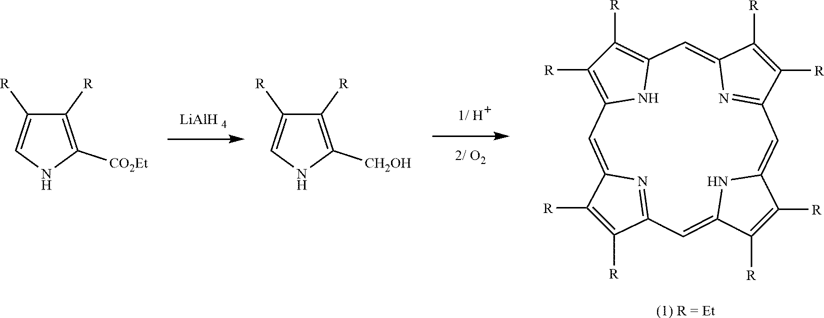

When the pyrrolic R groups are not identical, acid catalyzed tetramerization of the pyrrole results in the initial formation of the unstable porphyrinogen, which has been shown to undergo acid catalyzed pyrrole “flipping” or “scrambling”. This results in the formation of up to four porphyrinogen isomers (Scheme 2) which, on oxidation with air, forms the corresponding porphyrin isomers (Types I - IV). As a result, the quest to find a suitable synthesis route to centrosymmetric porphyrins, such as etioporphyrin-I (Scheme 2; type I), from mono-pyrrolic precursors has spurred several publications on the topic.

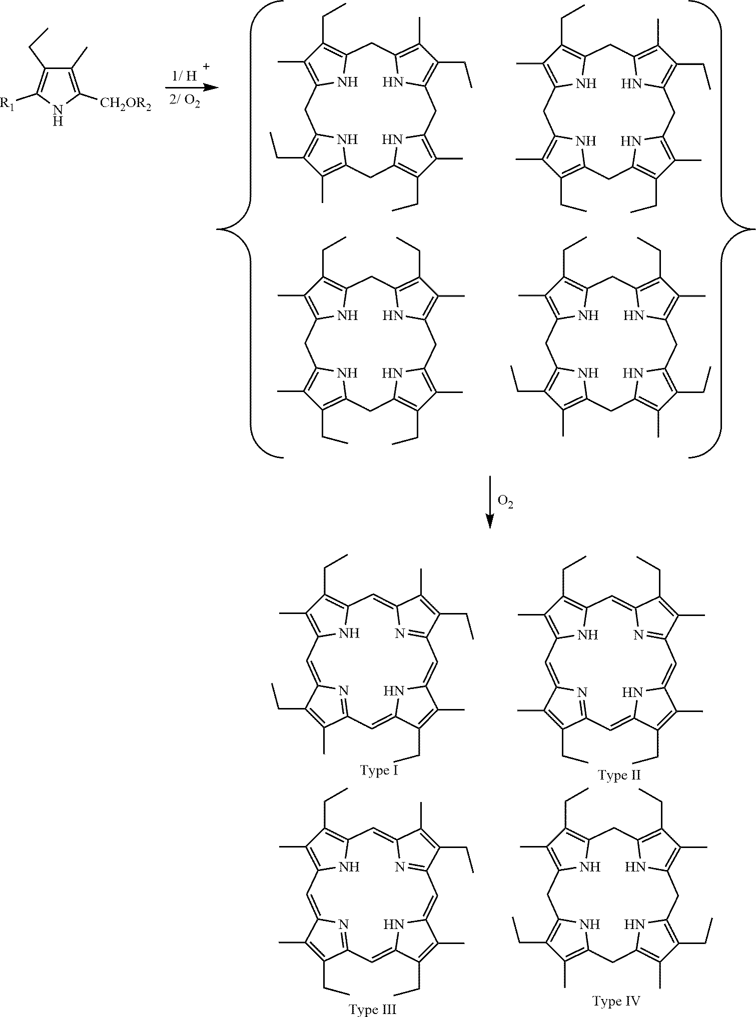

The porphyrinogen isomerization proceeds rapidly when the substituents are electron-donating groups such as alkyl or aryl groups. N. Ono and K. Maruyama (Chem. Letters, 1237-1240, 1989) reported that the isomerization during cyclization can be minimized in the heterogeneous reaction using silica gel as an acid catalyst. The reaction gave etioporphyrin-I in 30% yield from 2-(hydroxymethyl)pyrrole (pyrrole, Scheme 2, R₁, R₂ = H) and about 95% of structurally isomeric purity. The ratio of isomers was determined by NMR signals of the protons at the mesa positions or the methyl protons. The analyses of mesa peaks by NMR is inherently difficult and overestimates the isomeric purity of the sample. (Smith K, Nguyen, LT, Tet. Lett., 37(40), 7177-7180, 1996). H. Kinoshita et al. (Bull. Chem. Soc. Japan, 65, 2660-2667, 1992) showed the tetramerization can also be carried out under almost neutral or basic conditions and the corresponding Type I porphyrins resulted in 60-89% of structurally isomeric purity by proton NMR, insufficient for commercialization. Callot and coworkers (Bull. Soc. Chim. Fr, 130, 625-629, 1993) used a variety of reaction conditions that ultimately gave unacceptable levels of etioporphyrin isomers in low yield.

Historically, pure type I porphyrin isomers have been manufactured on small scales from brominated dipyrromethenes. The route to the synthesis of etioporphyrin-I using these intermediates is shown in Scheme 3.

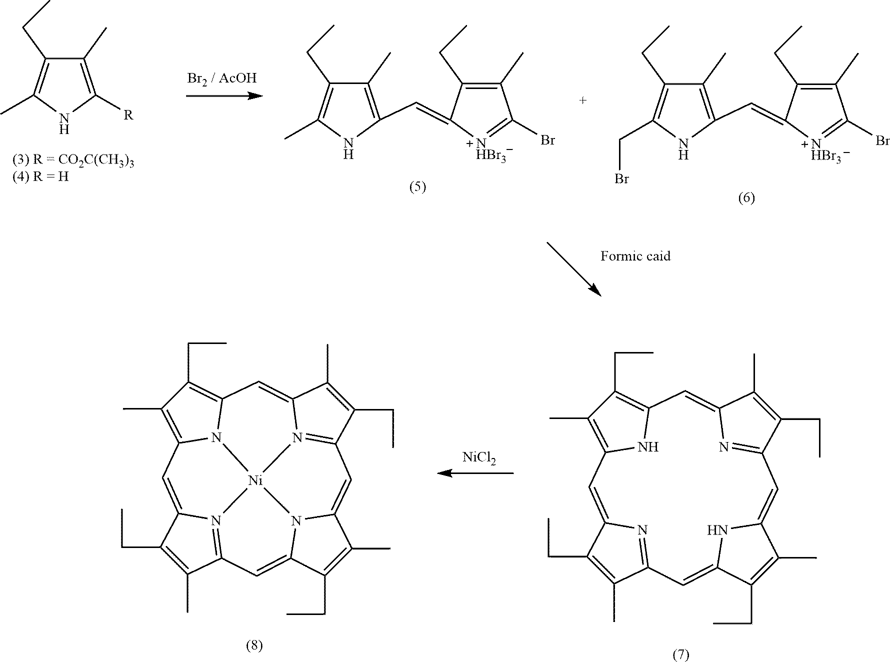

In the most common procedure, performed on relatively small scales (< 100 g), tert-butyl 4-ethyl-3,5-dimethyl-2-pyrrolecarboxylate (3) is reacted with an excess of bromine in acetic acid to give a mixture of brominated dipyrromethanes (5) and (6) in a 2/8 ratio (Paine, J.B., Hiom, J., Dolphin, D, J. Org. Chem., 53, 2796-2802, 1988. The dipyrromethene mixture is generally refluxed in formic acid to give etioporphyrin I in 20-35% yield. Alternatively, kryptopyrrole (4) has been reported to produce the dipyrromethenes (5) and (6) in a 2:3 ratio, although the authors warned that reactions over 10 g were not advisable (Rislove, D.J., O’brien, A.T., Sugihara, J.M., Journal of Chemical Engineering Data, 13(4), 588-590f. The monobromo-dipyrromethene (5) may be separated as a purple powder from the reaction mixture by dissolution with chloroform. The dibrominated dipyrromethene (6) is an oily residue that is only induced to crystallize with difficulty. Both brominated dipyrromethenes have been used to synthesize etioporphyrin-I. In each case the formed etioporphyrin-I is generally isolated via chromatography on silica.

In previous attempts to generate large quantities of etioporphyrin I for the large scale manufacturing of SnEt2, much of the chemistry outlined above has been repeated on large scale (> 1 kg) and have found that none of the reported processes are feasible from a large-scale commercial manufacturing viewpoint. There are several reasons for this. First, the bromination of tert-butyl 4-ethyl-3,5-dimethyl-2-pyrrolecarboxylate (3) in acetic acid is only feasible on scales < 100 g. Reactions larger than this results in a marked decrease in yield of the brominated dipyrromethenes under a variety of reaction conditions. In addition, at scales greater than 100 g the mono and dibrominated products form an oily reaction mixture that block almost every filter design making it virtually impossible to filter or isolate the solids. The viscous, highly acidic fumacious (HBr), solutions are difficult to work with or manipulate and represent a real workplace hazard.

To obtain reliable yields of the dipyrromethenes as a readily filterable solid, the bromination reaction needed to be performed at scales less than 100 g. Thus, to achieve etioporphyrin-I on scales > 100 g, multiple 100 g bromination reactions were required to supply enough dipyrromethene. Such batch processing makes the procedure inefficient and costly.

In addition to the problems associated with the isolation of the dipyrromethenes, another difficulty in the syntheses of etioporphyrin I is the work up and purification procedures for isolating etioporphyrin-I. The coupling reaction of the dipyrromethenes (see Scheme 3, (5) and (6)) is carried out in refluxing formic acid. Generally, this gives a tarry, black reaction mixture that yield about 20-35% of etioporphyrin-I after extensive chromatography. On small scales (1-40 g of dipyrromethenes), dissolution of the tarry reaction mixture in chloroform, followed by neutralization and chromatography on large amounts of silica affords the desired porphyrin. Unfortunately, the solubility of etioporphyrin-I in chloroform is extremely low (<10 mg / ml), hence large volumes of solvents and large amounts of silica gel are required to effect separation of etioporphyrin-I from the tarry byproducts. During this process it is not uncommon for etioporphyrin-I to precipitate from solution onto the silica gel, making column chromatography (even on small scale (10 g)) difficult. Clearly, such a process is not feasible economically on large scale.

The metallation of etioporphyrin with nickel to form nickel etioporphyrin-I has been known in the literature for many years. Generally, etioporphyrin-I is dissolved in dimethylformamide or acetic acid and refluxed with either nickel acetate or nickel chloride to give the required porphyrin. Unfortunately, the solubility of etioporphyrin I in either solvent is low (even at reflux) and hence generally large volumes of DMF or acetic acid are required to effectively metallate the porphyrin so that no free base etioporphyrin I remains. In a typical small-scale reaction, etioporphyrin-I (1 g) required about 500 mL of DMF to effect complete metallation of the porphyrin. Translating this to larger scales (1 kg) one can see instantly that very large volumes of solvents would be required (500 L) to effectively metallate the porphyrin. This loading ratio is unacceptable from a large-scale manufacturing viewpoint.

A need exists for improved methods for preparing nickel (II) etioporphyrin-I and related derivatives. The present invention seeks to fulfill this need and provides further related advantages.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

The present invention provides improved methods for the production of nickel (II) etioporphyrin-I.

In one embodiment, the invention provides a method for producing nickel (II) etioporphyrin-I, comprising;
(a) brominating kryptopyrrole to provide monobromo-dipyrromethene, wherein the bromination is carried out in the presence of ethyl acetate;
(b) cyclizing monobromo-dipyrromethene to provide etioporphyrin-I; and
(c) metallating etioporphyrin-I with a nickel (II) salt to provide nickel (II) etioporphyrin-I.

In another embodiment, the invention provides a method for producing nickel (II) etioporphyrin-I, comprising;
(a) brominating kryptopyrrole to provide monobromo-dipyrromethene;
(b) cyclizing monobromo-dipyrromethene to provide etioporphyrin-I, wherein the etioporphyrin-I is precipitated from solution using a dimethylformamide/acetone solvent; and
(c) metallating etioporphyrin-I with a nickel (II) salt to provide nickel (II) etioporphyrin-I.

In a further embodiment, the invention provides a method for producing nickel (II) etioporphyrin-I, comprising;
(a) brominating kryptopyrrole to provide monobromo-dipyrromethene;
(b) cyclizing monobromo-dipyrromethene to provide etioporphyrin-I; and
(c) metallating etioporphyrin-I with a nickel (II) salt to provide nickel (II) etioporphyrin-I, wherein the metalation is carried out in N-methylpyrrolidone.

In yet another embodiment, the invention provides a method for producing nickel (II) etioporphyrin-I, comprising;
(a) brominating kryptopyrrole to provide monobromo-dipyrromethene, wherein the bromination is carried out in the presence of ethyl acetate;
(b) cyclizing monobromo-dipyrromethene to provide etioporphyrin-I, wherein the etioporphyrin-I is precipitated from solution using a dimethylformamide/acetone solvent; and
(c) metallating etioporphyrin-I with a nickel (II) salt to provide nickel (II) etioporphyrin-I, wherein the metalation is carried out in N-methylpyrrolidone.

In another aspect, the invention provides nickel (II) etioporphyrin-I prepared by the above methods.

### DETAILED DESCRIPTION

The present invention provides improved methods for the production of nickel (II) etioporphyrin-I.

In one embodiment, the invention provides a method for producing nickel (II) etioporphyrin-I, comprising;
(a) brominating kryptopyrrole to provide monobromo-dipyrromethene, wherein the bromination is carried out in the presence of ethyl acetate;
(b) cyclizing monobromo-dipyrromethene to provide etioporphyrin-I; and
(c) metallating etioporphyrin-I with a nickel (II) salt to provide nickel (II) etioporphyrin-I.

In another embodiment, the invention provides a method for producing nickel (II) etioporphyrin-I, comprising;
(a) brominating kryptopyrrole to provide monobromo-dipyrromethene;
(b) cyclizing monobromo-dipyrromethene to provide etioporphyrin-I, wherein the etioporphyrin-I is precipitated from solution using a dimethylformamide/acetone solvent; and
(c) metallating etioporphyrin-I with a nickel (II) salt to provide nickel (II) etioporphyrin-I.

In a further embodiment, the invention provides a method for producing nickel (II) etioporphyrin-I, comprising;
(a) brominating kryptopyrrole to provide monobromo-dipyrromethene;
(b) cyclizing monobromo-dipyrromethene to provide etioporphyrin-I; and
(c) metallating etioporphyrin-I with a nickel (II) salt to provide nickel (II) etioporphyrin-I, wherein the metalation is carried out in N-methylpyrrolidone.

In yet another embodiment, the invention provides a method for producing nickel (II) etioporphyrin-I, comprising;
(a) brominating kryptopyrrole to provide monobromo-dipyrromethene, wherein the bromination is carried out in the presence of ethyl acetate;
(b) cyclizing monobromo-dipyrromethene to provide etioporphyrin-I, wherein the etioporphyrin-I is precipitated from solution using a dimethylformamide/acetone solvent; and
(c) metallating etioporphyrin-I with a nickel (II) salt to provide nickel (II) etioporphyrin-I, wherein the metalation is carried out in N-methylpyrrolidone.

In certain embodiments of the above methods, the methods further comprise purifying the etioporphyrin-I by an acid precipitation process using trifluoroacetic acid/dichloromethane and triethylamine.

In certain embodiments of the above methods, the method is carried out in a continuous stirred tank reactor.

The methods of the invention are capable of producing nickel (II) etioporphyrin-I in multi-kilogram quantities.

Three areas of improvement in the preparation of nickel (II) etioporphyrin-I were evaluated: (1) formation and isolation of the dipyrromethene; (2) formation and isolation of etioporphyrin-I; and (3) formation and isolation of nickel (II) etioporphyrin-I.

### Formation and Isolation of the Dipyrromethene (5)

The oily nature of the dibromodipyrromethene (6) has been recognized in the literature and is clearly problematic from a manufacturing / isolation viewpoint. Sintered or porous filters and centrifuges work well when products are crystalline. The brominated dipyrromethenes generated in the reaction of kryptopyrrole with bromine in acetic acid have a thick oily consistency. The reaction mixture is highly hygroscopic and it appears that the longer the brominated dipyrromethenes are exposed to acetic acid the more oily the reaction products become. This in turn makes them more difficult to isolate. Attempts to isolate and use the crude brominated reaction mixture in acetic acid were not fruitful. At scales greater that 2 kg, filtration of the oily brominated dipyrromethenes was found to be exceptionally problematic and if achieved formed unacceptable yields of etioporphyrin I in subsequent steps, most probably due to a lower yield of the dipyrromethenes. Regardless of the mode of addition of reactants, time of addition and temperature, the reactions proved to be problematic with respect to the isolation of the brominated dipyrromethenes.

It became clear that the problem in the isolation of the dipyrromethenes lies with the formation of the oily dibromo-dipyrromethene (6). Smith (Smith, K., Tet. Lett., (25), 2325-2328, 1971) had reported that the mono-brominated dipyrromethene (5) efficiently forms etioporphyrin-I in yields up to 60%. The focus of the development changed to center on the isolation of the dipyrromethene (5), which in its pure form, is a readily isolatable non-hydroscopic crystalline purple solid. During the course of development, dipyrromethene (5) was determined to be virtually insoluble in ethyl acetate, while the dibrominated dipyrromethene (6) is freely soluble. This afforded a route to isolate the desired dipyrromethene (5) from the reaction mixture. Reactions comprising a mixture of (5) and (6) were slurried in ethyl acetate and readily filtered to give (5) pure. The mother liquors contained the dibrominated dipyrromethene (6) and tarry by-products that were generally discarded. It occurred to us that perhaps the bromination reaction of kryptopyrrole could be performed in ethylacetate. Several authors have undertaken the bromination reaction of kryptopyrrole or tert-butyl 4-ethyl- 3,5-dimethyl-2-pyrrolecarboxylate (3) in solvents such as ether, chloroform or 1,2- dichloroethane. In these instances, the major product of the reaction is generally the dibrominated dipyrromethene (6). Surprisingly, the bromination of kryptopyrrole in ethyl acetate proceeds very smoothly with the desired mono-bromodipyrromethene precipitating virtually instantaneously from the reaction mixture. The solid is readily filtered and washed with ethyl acetate.

Reactions were undertaken to ascertain whether or the reaction could be scaled to the multiple kilogram level. Batch reactions performed at 1, 2, 4, or 8 kg of kryptopyrrole starting material successfully generated the mono-bromodipyrromethene, which was easily isolated using commercially available filtration equipment.

The immediate precipitation of the mono-bromodipyrromethene (5) from the ethyl acetate solution led to investigate the possibility of reacting kryptopyrrole and bromine in ethyl acetate using a continuous flow reactor. It was envisaged that such a process would enable tens of kilograms of the mono-bromodipyrromethene (5) to be produced in less than a day. This proved to be the case. In fact, the limitation of the process was building large enough filters to collect the brominated dipyrromethene. Yields from the reactor approach 90%. The filter design enables drying of the dipyrromethene with a continuous flow of air.

### Isolation of Etioporphyrin-I

Etioporphyrin-I is formed in 20-35% yield by refluxing the mono bromodipyrromethene (5) in formic acid. The reaction produces copious amounts (65-80%) of black, tarry viscous reaction by-products which makes isolation of the etioporphyrin I extremely difficult. Literature methods for the isolation of the porphyrin (Porphyrins and Metalloporphyrins, K. Smith Ed, p766, 1972) involving removal of the formic acid, followed by dissolution in hot chloroform, neutralizing the organic layer with sodium bicarbonate solution, evaporation of the organic layer and precipitation of the porphyrin from methylene chloride/ methanol, was found to be more of an art than a science and was not feasible on large scales.

It has also been found that the etioporphyrin-I dihydrobromide can easily precipitate out from N,N-dimethyl formamide (DMF)/acetone and leave most of the impurities in solution. This crude etioporphyrin-I dihydrobromide can be further purified by trifluoroacetic acid (TFA)/methylene chloride and triethyl amine (TEA). After nickel insertion in DMF, it gives 190 g of nickel etioporphyrin-I (8) with essential 95% pure.

The present invention provides an efficient method for manufacturing dipyrromethene (5), etioporphyrin-I (7), and nickel etioporphyrin-I (8) with high quality. In the examples that follow, there is disclosed the procedures of syntheses of nickel etioporphyrin-I (8) or the uses as starting material for SnEt2 syntheses.

Surprisingly, based on the results work by Rislove and coworkers, the bromination of kryptopyrrole (4) in acetic acid was found to be slightly better than using the pyrrole (3), and could be performed on larger than 100 g scales. In fact, reproducible yields of the brominated dipyrromethane were achieved at scales up to 1-2 kg of kryptopyrrole. At this scale the dipyrromethenes produced could be filtered slowly. Attempts to brominate kryptopyrrole at 4, 8 and 10 kg scales lead to severe filter blockage problems due to the oily nature of the resultant dipyrromethenes (5) and (6) and resulted in lower yields of the dipyrromethenes. This translated into even lower yields of the porphyrin, etioporphyrin I.

The etioporphyrin-I dihydrobromide formed in formic acid has different solubility in various organic solvents, so as impurities. Applicants had tried various solvents and found that acetone, methanol, DMF and ethyl alcohol dissolve impurities and precipitate etioporphyrin-I dihydrobromide out well. Among them acetone has the least solubility toward etioporphyrin-I dihydrobromide. The crude etioporphyrin-I dihydrobromide from acetone precipitation can be further purified by dissolving in TFA / methylene chloride, filtered off any solid impurities, then neutralized with TEA. Nickel insertion is then carried out in DMF with nickel (II) chloride hexahydrate. The present invention provides a convenient means for manufacture nickel etioporphyrin-I (8) from readily available starting materials.

The following examples are provided for the purpose of illustrating, not limiting the invention.

### Example 1

### Synthesis of 4-Acetyl-2-Ethoxycarbonyl-3.5-Dimethylpyrrole (20)

The synthesis of 4-acetyl-2-ethoxycarbonyl-3,5-dimethylpyrrole (20) is schematically illustrated in Scheme 6 below.

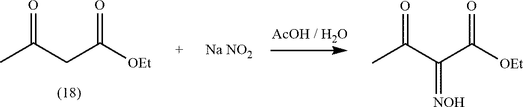

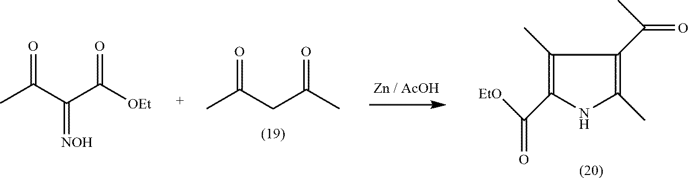

To a 22 L jacketed round bottom flask equipped with a 4-neck head, an overhead mechanical stirrer, an addition funnel, a thermocouple and cooling/heating circulator, was added ethyl acetoacetate (18) (1960 ml, 15.4 moles) and glacial acetic acid (7 L). A solution of sodium nitrite (1230 g, 15.0 moles) in warm water (1.8 L) was added dropwise to the stirred solution so as to maintain the temperature below 12° C. After all the sodium nitrite solution has been added, the mixture was stirred for a further 2 hours and then left to warm to room temperature with stirring overnight.

Into the above reaction mixture cooled to about 0-10° C. was added 2,4-pentanedione (19) (1780 ml, 17.3 moles) all at once. To the solution was added zinc dust (1900 g, 29.1 moles) in portions so that the temperature was maintained below 60° C. After the addition was complete, the mixture was heated at 90° C. for 2 hours, after which time all the excess Zn had dissolved. The hot solution was then slowly poured into 30 L of ice water with stirring. The crude 4-acetyl-2-ethoxycarbonyl-3,5-dimethylpyrrole (20) which precipitated was collected by filtration and thoroughly washed with water, then vacuum dried to give 2210 g of 4-acetyl-2-ethoxycarbonyl-3,5-dimethylpyrrole (20).

### Example 2

### Synthesis of 2-Ethoxycarbonyl-4-ethyl-3,5-dimethylpyrrole (21)

The synthesis of 2-ethoxycarbonyl-4-ethyl-3,5-dimethylpyrrole (21) is schematically illustrated in Scheme 7 below.

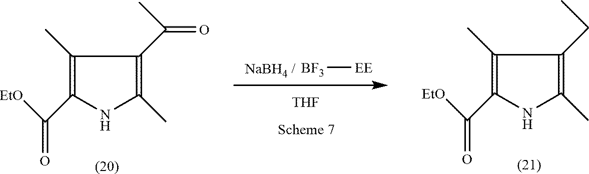

Into a 22 L jacketed round bottom flask equipped with an overhead mechanical stirrer, an addition funnel, a condenser, a thermocouple, a cooling / heating circulator and argon, was placed 4-acetyl-2-ethoxycarbonyl-3,5-dimethylpyrrole (20) (2200 g, 10.5 moles) and tetrahydrofuran (9 L). To the stirred solution was added sodium borohydride (725 g, 19.2 moles). The mixture was stirred for 1 hour at room temperature and then cooled to 5° C. Boron trifluoride etherate (2150 ml, 17.5 mole) was added dropwise so as to maintain the temperature at 10° C. After the addition was complete, the mixture was stirred for a further 2-3 hours after which time the reaction solution was checked by TLC for the absence of starting material. An excess of glacial acetic acid was then cautiously added until gas evolution ceased, while maintaining the temperature below 27° C. The mixture was then transferred to a container (30 L) and water (2 L) was added with stirring. The organic layer is filtered to remove boric acid, which was washed with methylene chloride (3 x 1 L). The combined THF/ether/CH₂Cl₂ solution was evaporated to dryness, water (2 L) was added and the crude 2-ethoxycarbonyl-4-ethyl-3,5-dimethylpyrrole (21) precipitate was collected by filtration. The solid was thoroughly washed with water and was used directly in the synthesis of kryptopyrrole (22).

### Example 3

### Synthesis of Kryptopyrrole (22)

The synthesis of kryptopyrrole (22) is schematically illustrated in Scheme 8 below.

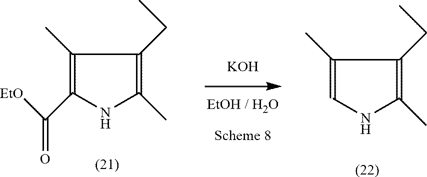

Into a 22 L round bottom flask equipped with an overhead mechanical stirrer and a condenser are placed, under argon, 2-ethoxycarbonyl-4-ethyl-3,5-dimethyl pyrrole (21) (from above (1950 g, based on 95% yield) and ethanol (5 L). The solution was stirred and warmed until the solid dissolved and then added a solution of sodium hydroxide (800 g, 20 moles) in water (650 ml). The mixture is heated under gentle reflux for 1 hour. Water (5 L) was added and the ethanol distilled off. The residue was allowed to cool to room temperature and then carefully acidified with glacial acetic acid (1145 ml, 20 mole) (A brown solid precipitated). The solution was heated under gentle reflux for 5 hours and allowed to cool to room temperature. The upper kryptopyrrole (22) was separated from the water and washed with water. The combined aqueous layers were extracted with methylene chloride (3 x 600 ml). The methylene chloride extracts are combined with the kryptopyrrole (22), dried over sodium sulfate, and the solvent removed. The crude kryptopyrrole (22) was obtained as a dark brown oil, which was vacuum distilled under argon at 92.5-94° C. / 18 mm). The light brown (or colorless) liquid was obtained in 88% yield.

### Example 4

### Synthesis of Nickel (II) Etioporphyrin-I (1)

The synthesis of nickel (II) etioporphyrin-I (1) is schematically illustrated in Scheme 9 below.

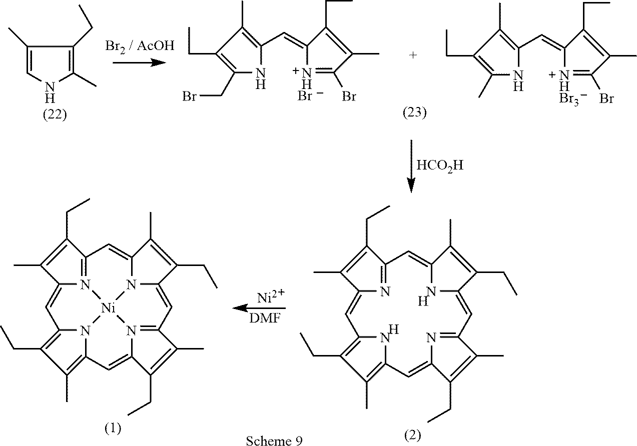

A 12 L 4-neck round bottom flask was equipped with a mechanical stirrer, a gas takeoff tube and two dropping funnels. AcOH (1 L) was added to the flask. Bromine (900 ml) and AcOH (1 L) were premixed and transferred to one of the dropping funnels. Kryptopyrrole (22) (1 kg) was transferred to the second dropping funnel. Kryptopyrrole (22) and the bromine solution was added at the same rate as quickly as possible while maintaining the temperature < 40° C. After the addition of the kryptopyrrole (22) and the first liter of the bromine solution, the remaining bromine solution was added as quickly as possible. After addition a further 2 L of AcOH was added and the solution was stirred for one hour at room temperature after which time a thick fine precipitate had formed. The solution was then filtered as rapidly as possible. The filtered dipyrromethene mixture (23) was layered with hexane overnight and refiltered the following day. The air dried dipyrromethene mixture (23) were transferred to a 12 L reaction vessel equipped with a mechanical stirrer and a gas take off tube and formic acid (7-8 L, 99%) was added. The thick reaction solution was warmed slowly to reflux whereby significant evolution of HBr gas occurred. The reaction was refluxed for 6 hours until the UV/visible spectrum showed the absence of dipyrromethene mixture (23). The solution was cooled overnight and the formic acid removed by rotary evaporation at < 50° C. to solid/liquid volume of about 1 L. DMF (700 ml) was added and toluene (500 ml) was added. The toluene was removed by rotary evaporation (toluene is used to remove formic acid). Toluene (500 ml) was added and again removed by rotary evaporation. Toluene (500 ml) was again added and removed by rotary evaporation. Acetone (1500 mL) was added and the solution cooled to room temperature with swirling. The solid porphyrin dihydrobromide was collected by filtration and washed with acetone until the filtrate was essentially colorless. The product was air dried to give 260 g of etioporphyrin-I dihydrobromide.

The etioporphyrin-I dihydrobromide (260-285 g) was dissolved in dichloromethane (2 L) and TFA (120 ml) with stirring. The solution was filtered and then neutralized with stirring using triethylamine (600 mL). The thick porphyrin precipitate was collected by filtration and washed well with methanol (1 L). This solid was dried under vacuum. Yield 165-190 g of etioporphyrin-I (2). The mother liquors were concentrated to about 500 mL and refiltered and washed with methanol to give a further 10 g of etioporphyrin-I (2).

Etioporphyrin-I (2) (300 g) was suspended in dimethylformamide (DMF) (17 L) and NiCl₂·6H₂0 (214.3 g) was added. The solution was refluxed overnight, whereby TLC showed the absence of starting material. DMF (8 L) was distilled from the reaction vessel and the solution cooled slowly to room temperature. The mass of nickel porphyrin crystals was collected by filtration and washed with methanol (1 L), hot water (1 L) and again with methanol (1 L). The solid was collected and vacuum dried to give 300 g of nickel etioporphyrin I (1).

### Example 5

### Procedure for the Production of Nickel (II) Etioporphyrin-1

In one aspect, the invention provides a three-step method for the preparation of nickel (II) etioporphyrin-1 by metallation of free base etioporphyrin-1, which is prepared from kryptopyrrole. The method is illustrated schematically below.

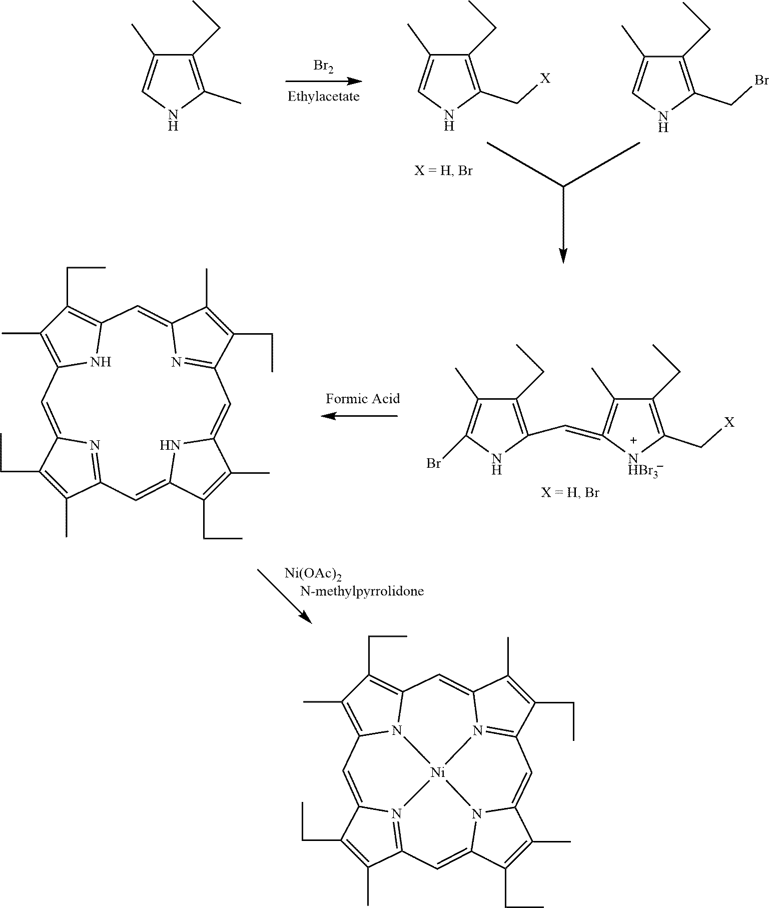

The following describes a representative three-step procedure for the production of nickel (II) etioporphyrin-1.

[Note: silicon grease should not be used on any glassware used in this process.]

### Step One: Monobromo-Dipyrromethene From Kryptopyrrole

In the first step, kryptopyrrole (KP) is reacted with bromine (Br₂) to provide the monobromo-dipyrromethene.

The reaction is undertaken in a continuous reactor produces the required dipyrromethene continuously. The kryptopyrrole and bromine are reacted above the surface of the ethyl acetate and the resulting mixture immediately precipitates the desired monobromo-dipyrromethene. The dipyrromethene is vacuumed removed from the reaction vessel to a vacuum filter (TSM filter) where it is pumped dry.

The following is an exemplary procedure.

### Dihydrobromide Intermediate Salt (monobromo-dipyrromethene)

1. A 5 L continuous stirred tank reactor (CSTR) is used which allows for reagents to be pumped in through the top and product solution to be removed through an overflow tube. The reactor is equipped with a cooling jacket, stir paddle, reagent inlet tubes, inert gas inlet line, and vacuum adapter port. The reaction is run under vacuum with an inert gas purge to remove excess HBr.

2. The overflow (outlet) tube of the CSTR is connected to the inlet of a pressure filter. The outlet of the pressure filter is connected to the vacuum system.

3. Charge the CSTR with ethyl acetate and stir. Cool the CSTR contents to between -10 and 0° C.

4. Start the vacuum system and adjust the pressure by adjusting the flowrate of inert gas into the system.

5. Begin pumping the reagents; bromine, kryptopyrrole, and ethyl acetate, into the CSTR. Maintain the temperature of the reaction mixture below 40° C.

6. Continue reagent addition until the desired batch size is reached. Maximum batch size is limited by the capacity of the filter being used.

7. Reduce the agitator speed and lower the overflow tube to empty the CSTR.

8. When the overflow tube has been lowered to the bottom of the CSTR, charge additional ethyl acetate to the CSTR and flush any remaining intermediate salts into the pressure filter.

9. Disconnect the pressure filter from the system and dry the contents by flowing inert gas through the filter.

### Step Two: Etioporphyrin-I From the Monobromo-Dipyrromethene

The cyclization of the monobromo-dipyrromethene to etioporphyrin I is carried out in refluxing formic acid. The product etioporphyrin-I is selectively precipitated from the reaction mixture through the use of acetone. Impurities such as mono-bromo etioporphyrin-I are removed from the crude etioporphyrin-I product by the use of an acid precipitation technique described below. Yields are typically 30%.

The following is are exemplary procedures.

### Etioporphyrin-1 Dihydrobromide

1. Add formic acid (4-5 kg HCOOH / kg KP) to the material in the filter. Stir the mixture vigorously and transfer the slurry to the reactor using inert gas pressure and/or vacuum.

2. Heat the slurry to 90 ± 5° C. Hold the mixture at ≥ 85° C. for 4 ± 0.5 hours.

3. Distill the mixture under vacuum until half of the formic acid has been removed.

4. Using vacuum, add toluene (3 - 3.5 kg toluene/ kg KP) to the reactor. Formic acid and toluene form an azeotrope that boils at about 38° C. at 26 inches Hg.

5. Continue the vacuum distillation of the mixture. As distillate is collected, add an equivalent volume of toluene to the reactor. The distillate will split into two layers. The volume of the formic acid should be measured. Once 75 - 85% of the formic acid has been collected, stop the distillation and allow the mixture to cool to ambient temperature.

6. Add acetone (1 - 3 kg acetone/ kg KP) and stir for 30 minutes. Filter the mixture and wash the solid filter cake with acetone (1 - 3 kg acetone / kg KP).

7. Dry the solids under vacuum to a constant weight.

### Freebase Etioporphyrin-1

1. Charge the reactor with methanol (0.86 kg MeOH / kg HBr salt) and etioporphyrin-1 dihydrobromide.

2. Add triethylamine (1.16 kg TEA / kg HBr salt). There is an exotherm associated with the neutralization of etioporphyrin-1 dihydrobromide, maintain the temperature below 50° C.

3. Allow the mixture to cool to room temperature. Filter the mixture and wash the solid filter cake with purified water (1.46 kg H₂O / kg HBr salt), followed by 1/1 v/v methanol/ water (1.30 kg solution / kg HBr salt), and then methanol (0.58 kg MeOH / kg HBr salt).

4. Dry the material under vacuum to a constant weight.

### Purification of Etioporphyrin-1

1. Charge the reactor with trifluoroacetic acid (2.25 kg TFA / kg etioporphyrin-1), formic acid (10.40 kg HCOOH / kg etioporphyrin-1) and methanol (3.91 kg MeOH / kg etioporphyrin-1). There is an exotherm associated with dissolution of methanol in the acid mixture.

2. After the solution has cooled to ambient temperature add the etioporphyrin-1 free base.

3. Add methanol rapidly to the mixture (11.74 kg MeOH / kg etioporphyrin-1). Monitor the purification by filtering an aliquot and sampling the filtrate. Determine the impurity level in the filtrate by HPLC.

4. When the impurity level in the liquid phase drops below 0.2%, filter the mixture through a high capacity pre-filter and then a hydrophobic 0.2 µm filter. Rinse the filtration apparatus with methanol.

5. Distill the mixture to about one-third of its original volume. The temperature of the reaction mixture at this point should be 65-75° C.

6. Allow the mixture to cool to ambient temperature and add triethylamine to neutralize any residual acid and adjust the pH (2 - 3 kg TEA / kg etioporphyrin-1). Allow the mixture to cool to ambient temperature again.

7. Filter the mixture and wash the solid filter cake with 1/1 v/v methanol/ water (5 kg solution / kg etioprophyrin-1) and then methanol (0.75 kg MeOH / kg etioporphyrin-1).

8. Dry the product under vacuum to a constant weight.

### Step Three: Nickel (II) Etioporphyrin-I From Etioporphyrin-I

Metallation of etioporphyrin I with a nickel (II) salt provides nickel (II) etioporphyrin-I. The solvent of choice is N-methylpyrrolidone, which has improved loading that is three times (3X) greater than for dimethylformamide (DMF).

The following is an exemplary procedure.

### Nickel (II) Etioporphyrin-1

1. Charge N-methyl-2-pyrrolidinone (Table I), etioporphyrin-1 (Table I), and nickel (II) acetate • 4 H₂O) (0.634 kg NiOAc • 4 H₂O) to the reactor.

2. Heat the mixture to the temperature specified in Table I and hold for at least 1 hour.

3. After one hour has elapsed (and each hour thereafter), sample the mixture. Allow the sample to cool to ambient temperature and filter. Wash the solids with a minimal amount of water and then methanol. Analyze the solid by HPLC. When the sample meets the specification (98%, SM 0.5%), allow the entire mixture to cool to ambient temperature.

4. Filter the mixture and wash the solid filter cake with purified water (2.4 kg H₂O /kg etioporphyrin-1) and methanol (1.9 kg MeOH/kg etioporphyrin-1).

5. Dry the product under vacuum to a constant weight.

TABLE 1

Loading data

| Temperature (° C) | Loading (moles/kg solvent) |
|---|---|
| 150 | 0.11 |
| 160 | 0.12 |
| 170 | 0.15 |
| 180 | 0.16 |
| 190 | 0.17 |

While illustrative embodiments have been illustrated and described, it will be appreciated that various changes can be made therein without departing from the spirit and scope of the invention.

1. A method for producing nickel (II) etioporphyrin-I, comprising;
(a) brominating kryptopyrrole to provide monobromo-dipyrromethene, wherein the bromination is carried out in the presence of ethyl acetate;
(b) cyclizing monobromo-dipyrromethene to provide etioporphyrin-I; and
(c) metallating etioporphyrin-I with a nickel (II) salt to provide nickel (II) etioporphyrin-I.

2. A method for producing nickel (II) etioporphyrin-I, comprising;
(a) brominating kryptopyrrole to provide monobromo-dipyrromethene;
(b) cyclizing monobromo-dipyrromethene to provide etioporphyrin-I, wherein the etioporphyrin-I is precipitated from solution using a dimethylformamide/acetone solvent; and
(c) metallating etioporphyrin-I with a nickel (II) salt to provide nickel (II) etioporphyrin-I.

3. A method for producing nickel (II) etioporphyrin-I, comprising;
(a) brominating kryptopyrrole to provide monobromo-dipyrromethene;
(b) cyclizing monobromo-dipyrromethene to provide etioporphyrin-I; and
(c) metallating etioporphyrin-I with a nickel (II) salt to provide nickel (II) etioporphyrin-I, wherein the metalation is carried out in N-methylpyrrolidone.

4. A method for producing nickel (II) etioporphyrin-I, comprising;
(a) brominating kryptopyrrole to provide monobromo-dipyrromethene, wherein the bromination is carried out in the presence of ethyl acetate;
(b) cyclizing monobromo-dipyrromethene to provide etioporphyrin-I, wherein the etioporphyrin-I is precipitated from solution using a dimethylformamide/acetone solvent; and
(c) metallating etioporphyrin-I with a nickel (II) salt to provide nickel (II) etioporphyrin-I, wherein the metalation is carried out in N-methylpyrrolidone.

5. The method of claim 1 further comprising purifying the etioporphyrin-I by an acid precipitation process using trifluoroacetic acid/dichloromethane and triethylamine.

6. The method of claim 1, wherein the method is carried out in a continuous stirred tank reactor.

7. The method of claim 1, wherein nickel (II) etioporphyrin-I is produced in multi-kilogram quantities.

8. (canceled)

9. The method of claim 2 further comprising purifying the etioporphyrin-I by an acid precipitation process using trifluoroacetic acid/dichloromethane and triethylamine.

10. The method of claim 2, wherein the method is carried out in a continuous stirred tank reactor.

11. The method of claim 2, wherein nickel (II) etioporphyrin-I is produced in multi-kilogram quantities.

12. The method of claim 3 further comprising purifying the etioporphyrin-I by an acid precipitation process using trifluoroacetic acid/dichloromethane and triethylamine.

13. The method of claim 3, wherein the method is carried out in a continuous stirred tank reactor.

14. The method of claim 3, wherein nickel (II) etioporphyrin-I is produced in multi-kilogram quantities.

15. The method of claim 4 further comprising purifying the etioporphyrin-I by an acid precipitation process using trifluoroacetic acid/dichloromethane and triethylamine.

16. The method of claim 4, wherein the method is carried out in a continuous stirred tank reactor.

17. The method of claim 4, wherein nickel (II) etioporphyrin-I is produced in multi-kilogram quantities.
